(19) United States
(12) Patent Application Publication
(10) Pub. No.: US 2012/0232578 A1
(43) Pub. Date: Sep. 13, 2012

(54) APPARATUS AND METHODS FOR CONTROLLING BLEEDING USING EXTERNALLY APPLIED PRESSURE

(75) Inventors: David E. Altobelli, Hollis, NH (US); Gregory R. Lanier, JR., Merrimack, NH (US); Stewart M. Coulter, Bedford, NH (US); Wayne R. Penn, Nashua, NH (US)

(73) Assignee: DEKA Products Limited Partnership, Manchester, NH (US)

(21) Appl. No.: 13/372,149

(22) Filed: Feb. 13, 2012

Related U.S. Application Data
(60) Provisional application No. 61/441,937, filed on Feb. 11, 2011.

(51) Int. Cl. A61B 17/135

(52) U.S. Cl. 606/202

(57) ABSTRACT

An apparatus and system is disclosed for applying a compressive force to the axilla of an individual to reduce or inter the flow of blood to the individual's upper extremity. An inflatable bladder may be mounted on a flexible plate and directed toward the apex of the axilla. The flexible plate may be attached to a flexible retention strap that can be wrapped around the individual's torso or over the individual's shoulder. The retention strap may be secured in a number of ways, and the flexible plate and attached strap provide enough stiffness to direct the expanding bladder toward the axilla. The expanded shape of the bladder may conform to the shape of the axillary recess, or an attached adjacent compression mass may be sufficiently flexible upon expansion of the bladder to conform to the shape of the axillary recess.

### RELATED APPLICATIONS

This application claims the benefit of U.S. Provisional Application Ser. No. 61/441,937, filed Feb. 11, 2011, which is incorporated herein by reference in its entirety.

### FIELD OF INVENTION

The present invention generally relates to devices and methods of controlling bleeding in an extremity at locations not readily amenable to the application of a tourniquet or similar device.

### BACKGROUND

One may occlude blood flow through any of a number of extremity arteries by applying direct pressure using one's hands or fingers to skin and soft tissue overlying the artery. However, operator fatigue and limited personnel resources may make it impractical to use this method for prolonged periods of time.

Tourniquets and existing direct-pressure devices can be effective in occluding arteries in certain locations, such as the forearm the radial artery), the upper aim (e.g., brachial artery), the lower leg (e.g., anterior or posterior tibial arteries), or the mid-to-lower thigh (e.g., femoral artery). However, if control of arterial flow in an extremity must be obtained more proximally near the torso, a tourniquet may be ineffective, and existing direct-pressure devices may be impractical to apply, or may fail to provide enough pressure directly over the target artery without causing excessive pressure to other nearby tissues.

The axillary artery at the apex of the axilla is an example of such a challenging location. Referring to FIG. 1, the axillary artery 2 is a continuation of the subclavian artery, and becomes the brachial artery 4 at the lateral edge of the pectoralis major muscle 6. The artery traverses the apex of the axilla 8 inferior to the anterior border of the deltoid muscle 10, and is covered in part by the pectoralis minor and more distally by the pectoralis major 6 muscles. It crosses anterior to the teres major muscle 12 and becomes more superficial and palpable in the axilla 8, just before becoming the brachial artery 4. it is about six inches in length at this location. The artery is curved with the arm hanging by the side of the chest, and becomes relatively horizontal by raising the arm to a right angle with the chest. Because of the topography of the axilla, it is more difficult to compress the axillary artery against the humeral head than it is, for example, to compress the femoral artery against the femoral head. Furthermore, the brachial plexus 14 is immediately adjacent to the artery, and is susceptible to injury from an excessive or widely distributed compressive force against the axillary artery.

The axilla 8 may be considered to be a roughly pyramidal or frustoconical space or recess, bordered anteriorly by the pectoralis major 6 and minor muscles and posteriorly by the subscapularis, teres major 12 and latissimus dorsi muscles. It is difficult to compress the axillary artery through the anterior wall of the axilla, which is formed by the pectoralis major 6 and minor muscles. The axillary artery 2 is more easily compressed directly within the axilla 8, directing the force toward the apex of the axillary recess, necessitating an inferior approach of the compressive force. Preferably, the compressive force should be applied relatively broadly within the axilla to address collateral blood flow around the axillary artery, and to reduce focal areas of excessive tissue pressure. Prior art devices better suited for other locations have used a contacting or compressing element that is constructed to maintain its shape over the skin pressure area. This may work adequately over certain types of surface anatomy (e.g., over the femoral artery in the groin). However, in locations such as the axilla, the location of the axillary artery in relation to the anatomy of the axillary recess may vary significantly from person to person. In this type of location, a contacting or compressive element should be soft enough to conform to the shape of an individual's axilla, and should be backed by an expanding element (such as a bladder) and a semi-rigid backing plate that can accommodate variations in the anatomy of the walls of the axilla, and the movements of the arm and shoulder that may occur after the device has been applied.

### SUMMARY OF INVENTION

In an embodiment, an apparatus for applying pressure to an individual's axilla comprises an inflatable bladder mounted on flexible plate, a flexible strap configured to support the plate in a position to allow an expandable surface of the inflatable bladder opposite the plate to face the axilla, wherein the flexible strap is of a length sufficient to wrap around the individual's torso or over one of the individual's shoulders. The apparatus may further comprise a compressing mass adjacent the expandable surface of the inflatable bladder, wherein the compressing mass is of a size and shape configured to apply direct pressure to skin at an apex of the individual's axilla. The expandable surface of the inflatable bladder may alternatively be configured to have a size and shape upon inflation to apply direct pressure to skin at an apex of the individual's axilla. The inflated shape of the expandable surface of the inflatable bladder may be frusto-conical or frusto-pyramidal. The compressing mass may comprise a flexible component configured to conform to the shape of an apex of the individual's axilla. The flexible strap may comprise hook-and-loop material to allow an end of the flexible strap to be secured to one or more surfaces of the flexible strap.

In another embodiment, a method of applying a compressive force to the axilla of an individual using a compressive device comprising an inflatable bladder mounted to a flexible base attached to a flexible strap may comprise facing an expandable surface of the bladder toward the individual's axilla, wrapping the flexible strap around the individual's torso or over the individual's shoulder, and inflating the inflatable bladder. The the compressive device may further comprise a compressing mass adjacent the expandable surface of the inflatable bladder, the method further comprising facing the compressing mass toward the individual's axilla. The the flexible strap may comprise hook-and-loop material, the method further comprising securing an end of the flexible strap to a portion of the hook-and-loop material after wrapping the flexible strap. The flexible strap may alternatively comprise a buckle, the method further comprising securing an end of the flexible strap by threading it through the buckle after wrapping the flexible strap. The method may further comprise inflating the inflatable bladder until blood flow through the axillary artery in the axilla is interrupted. The interruption of blood flow in the axillary artery may be detected by monitoring the brachial pulse, radial pulse, ulnar pulse, or by pulse oximetry of a portion of the individual's upper extremity.

In another embodiment, a system for reducing blood flow in a person's axilla by the application of pressure to skin and soft tissue in the axilla may comprise an inflatable bladder mounted on a flexible plate, an expandable portion of the bladder configured to expand toward an apex of the axilla, a flexible strap to which the plate is attached, the strap being of a length sufficient to wrap around the person's torso or over the person's shoulder, a releasable valve connected to the bladder, the releasable valve configured to connect to a manual or electromechanical pump for inflating the bladder, wherein the inflatable bladder is configured to expand toward the apex of the axilla. The system may further comprise a compressing mass adjacent the expandable portion of the bladder, wherein the compressing mass is configured to generally conform to the shape of the apex of the axilla upon inflation of the bladder. The expandable surface of the inflatable bladder may be configured to have a size and shape upon inflation to apply direct pressure to skin at an apex of the individual's axilla. The inflated shape of the expandable surface of the inflatable bladder may be frusto-conical or frusto-pyramidal. The the amount of expansion of the bladder may be adjustable by operation of the releasable valve to release incremental amounts of air from the bladder. Operation of the electromechanical pump may be regulated to limit the inflation of the bladder to a pre-determined amount above the amount of inflation required to interrupt blood flow in an axillary artery within the axilla. The the interruption of blood flow may be detected by monitoring a brachial, radial or ulnar pulse, or by monitoring pulse oximetry in a portion of the person's upper extremity. The system may further comprise a controller configured to receive a signal from a pulse sensor or oximetry sensor, and may be configured to control the electromechanical pump and releasable valve to regulate the amount of inflation of the bladder.

### BRIEF DESCRIPTION OF THE DRAWINGS

FIG. 1 is an illustration of the anatomy of the shoulder and axilla.

FIG. 2 is an illustration of the placement of an inflatable bladder and compressing mass in the

FIG. 3 shows an example of the material that may comprise a compressing mass.

FIG. 4 is a schematic representation of an embodiment of a compressing MASS and expandable member.

FIG. 5 shows the placement of a flexible retention strap in relation to the inflatable bladder and compressing mass.

FIG. 6 illustrates an alternate placement of a flexible retention strap.

FIG. 7 illustrates another arrangement of the flexible retention strap over a person's torso and shoulders.

FIGS. 8A and 8B show examples of an inflatable bladder and insufflation devices.

FIG. 9 shows an embodiment of the disclosed invention.

FIG. 10 shows an embodiment of an inflatable bladder in a collapsed and inflated state.

FIG. 11 illustrates how an embodiment of the disclosed invention may be folded for storage.

FIG. 12 illustrates an embodiment of the invention using an automated monitoring and inflation system.

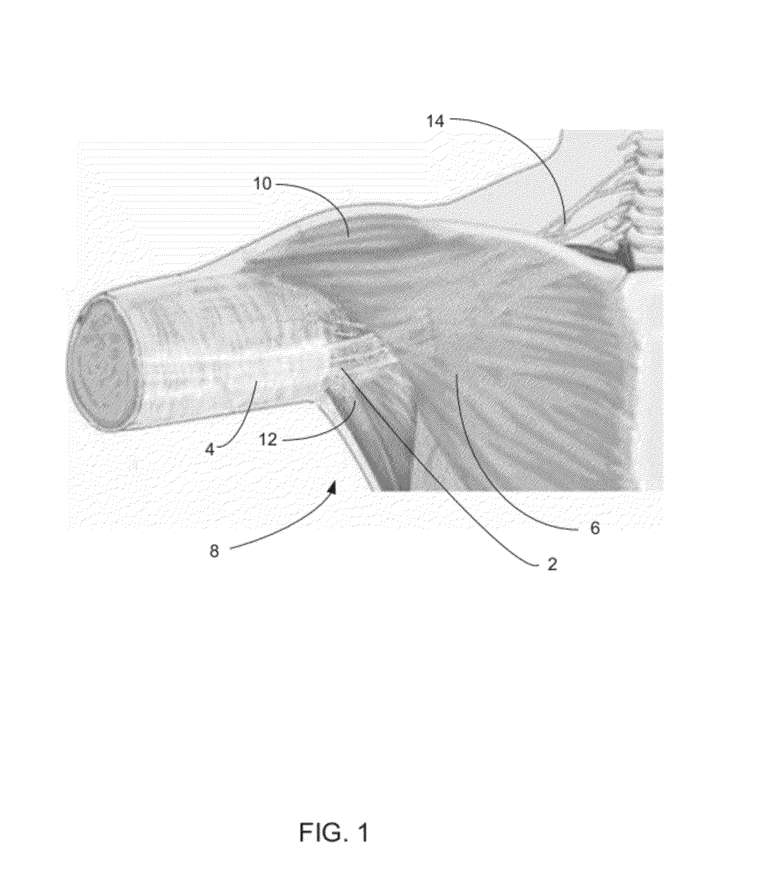

### DETAILED DESCRIPTION

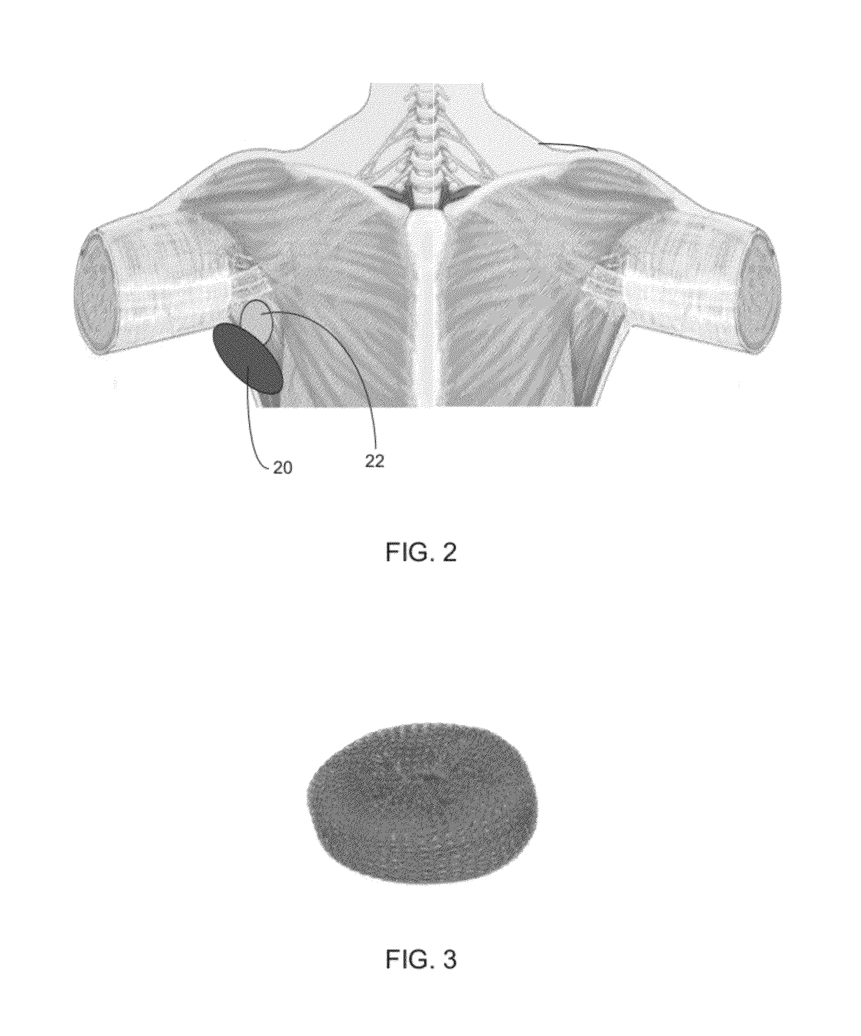

Referring to FIG. 2, in an embodiment, an inflatable bladder 20, which may optionally act on a compressing or contacting element or mass 22, can be positioned inferiorly over the axilla, and held in place by a strap or sling crossing the upper chest and wrapping around the shoulder contralateral (opposite side) or ipsilateral (same side) to the axilla in which pressure is to be applied. The unique and complex anatomy of the shoulder and axilla may render the use of a rigid frame member to hold the bladder and/or occluder impractical. Preferably, the contacting element or compressing mass is moldable or conformable to the shape of a person's axilla sufficiently to apply pressure directly over the skin at the apex of the axilla. The lateral deflection or expansion of the compressing mass (i.e., in directions other than toward the apex of the axilla or the underlying axillary artery) may be limited by the anatomical structures forming the walls that bound the axillary recess.

As shown in FIG. 2, a compressing mass 22 preferably may be placed adjacent the inflatable bladder 20, in this example the mass being positioned within the axilla and helping to focus the pressure generated by the inflated bladder directly against the soft tissue overlying the target artery (in this example, the axillary artery). In some cases, the distribution of pressure generated by the bladder alone results in the arterial occluding pressure being inefficiently applied, which would require higher bladder pressures, and which would in turn cause unnecessarily excessive compression of soft tissue near the target artery. The compressing mass is preferably partially elastic and has a contacting surface that is convex or semi-spheroid. various aspects, it may be solid or hollow; and various portions may be rigid, semi-rigid and elastic. Alternatively, the skin-contacting portion may be soft and conformable to the recesses and uneven surface topography of the axillary skin and soft tissue to be compressed. In one aspect, the compressing mass 22 may be of a size, shape and resiliency approximating that of a tennis ball or racquetball, or of a portion thereof. Alternatively, it may be of a size, shape and resiliency of a solid rubber sphere about the size of, or somewhat larger than a lacrosse ball or a field hockey ball, for example.

Other compressing masses may consist of plastic mesh material bundled together, not unlike a plastic mesh bundle, as shown in FIG. 3. This material has the ability to conform to an irregular surface topography upon application of modest compression. Once it has been compressed to conform to the topography of the skin surface, it is then capable of transmitting to the skin any additional compressive force against the mesh material.

The compressing mass may also comprise a collection of compressible minibeads or microbeads enclosed within an airtight flexible bag. A valved port on the bag allows for application of a vacuum to the contents of the bag in a manner similar to vacuum immobilization splints. A vacuum immobilization splint generally consists of an airtight flexible envelope having a valved port through which air in the envelope may be evacuated. The envelope contains compressible beads such as pliable expanded polymer or polystyrene beads, usually about 2 mm in diameter. The beads may be microspheres of polystyrene or Styrofoam-type material. Once evacuated, the envelope constricts around the beads and compresses them. Friction between beads inside the envelope and between the beads and the inner surface of the envelope solidifies the shape of the evacuated envelope in the form in which it was approximately placed immediately prior to the application of the vacuum. The density of the beads may vary. Higher density beads may provide greater resistance to collapsing of the beads, which helps to minimize any alteration of the initial shape in which the envelope is placed before vacuum application. An advantage of a vacuum immobilization occluder bag is the ability to mold the bag prior to evacuation to conform it to the specific dimensions of each patient's individual surface anatomy. Upon evacuation of the bag, the occluder becomes rigid and may be compressed by an adjacent bladder to achieve an efficient transfer of compressive force against the target artery under the surface of the skin.

In another embodiment, the compressing mass may comprise a soft, conformable but cohesive silicone elastomeric gel, made, for example from polyorganosiloxane compounds. This material generally consists of highly crosslinked polysiloxane networks, swollen with a polydimethylsiloxane (PDMS) fluid, forming a semi-solid mass that tends to retain its form without having to be enveloped in a fabric or other containment structure.

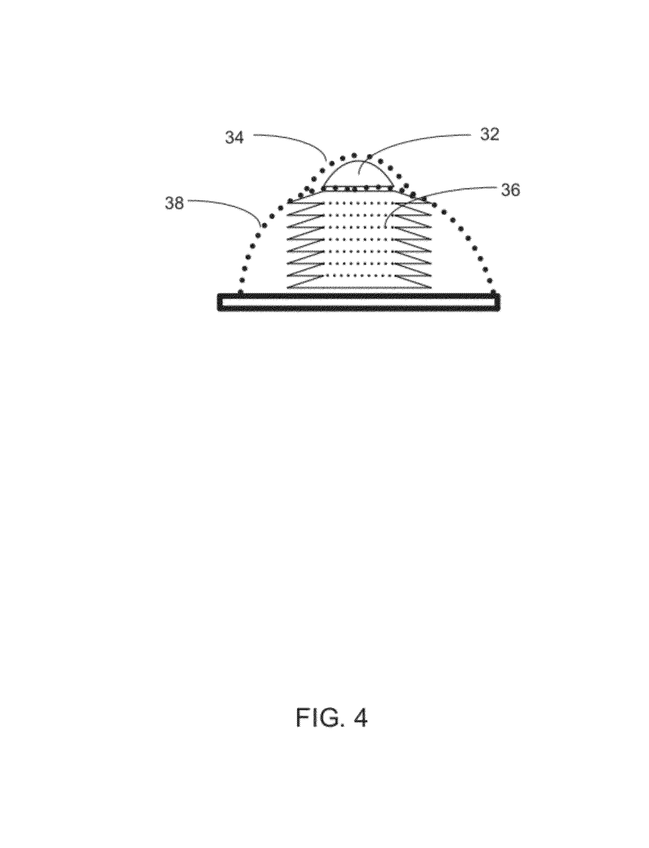

As schematically illustrated in FIG. 4, in yet another embodiment, the compressing mass 32 may be inserted into a fabric pocket 34 overlying an inflatable bladder 36, which may itself optionally be secured within a fabric pocket 38 of sufficient capacity to accommodate inflation and expansion of the bladder. In one aspect, the fabric material may be elastic and may conform more closely to the dimensions of the bladder, the elasticity of the fabric allowing it to stretch to accommodate the expansion of the bladder. If a more directed force on the compressing mass is desired, a bellows-type or accordion-type inflatable bladder 36 may be used.

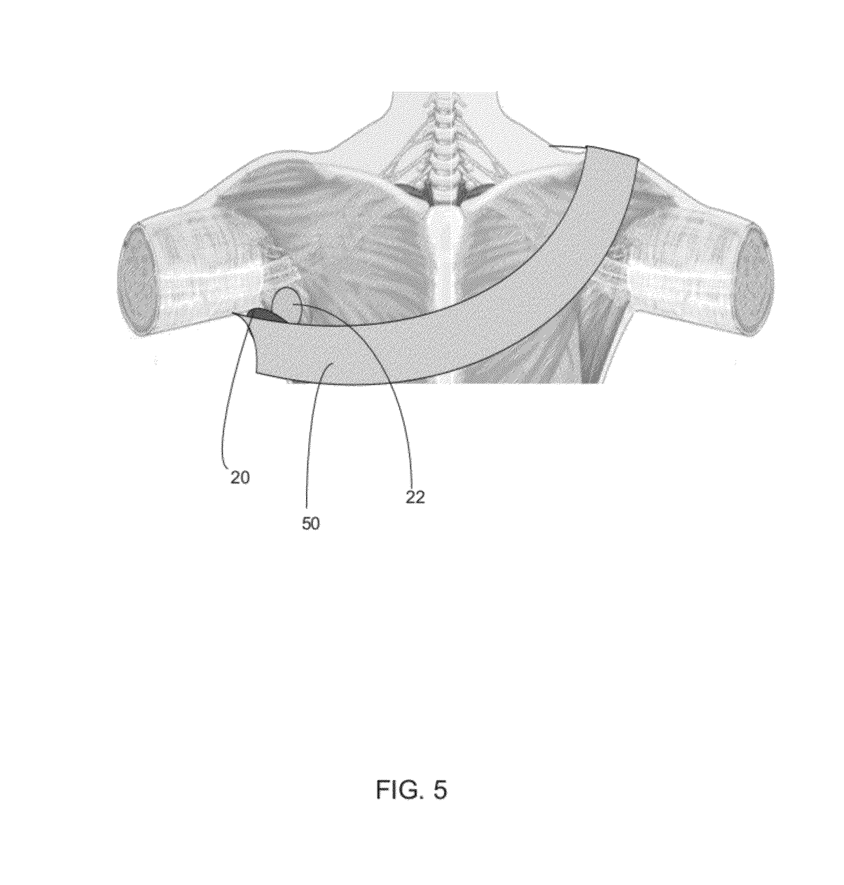

As shown in FIG. 5, the bladder and occluder assembly may be mounted on a restraining or retention strap 50 that can be wrapped around the torso in a number of possible patterns. The strap 50 may be constructed of elastic material, such as spandex or Spandura® a combination of non-woven nylon (e.g., Cordura®) and, for example, Lycra® that combines properties of durability with elasticity. The strap 50 may also be constructed of neoprene, for example, which provides strength, flexibility with more modest elasticity. Alternatively, a more durable and less elastic fabric such as a nylon web may be used, the thickness of which may be selected to provide adequate strength with enough flexibility to bend or fold around the neck or shoulder. In an embodiment, a hook-and-loop fastener material (e.g., Velcro®) may be sewn onto the strap material, with one end having the loop surface, and the other end having the hook surface. Alternatively, a Velcro®-sensitive neoprene may be selected, to permit the strap to be cinched around the torso and axilla, and be readily secured by fastening a hook surface to bridge the loop sides of the Velcro®-sensitive neoprene wrap.

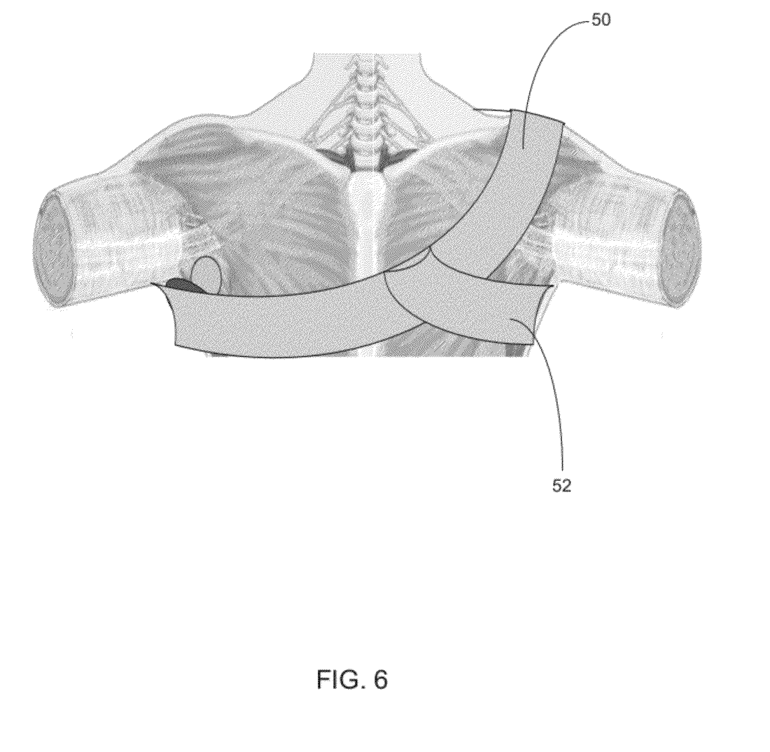

To improve the stability of the retention strap, it may be constructed to wrap around e torso in a number of patterns, including, for example, a crossing pattern over the opposing shoulder 50 and under the opposing axilla 52, as shown in FIG. 6. The strap pattern may be stabilized, for example with the use of bridging hook-and-loop strips. In another aspect, a wide buckle may be used, attached near one end of the strap and allowing the other end of the strap to be threaded through the buckle and cinched to the desired tension. The buckle may include a row of releasable cam features along its width to grip the cinched strap and hold it at the desired tension, and to prevent the strap from bunching up within the buckle.

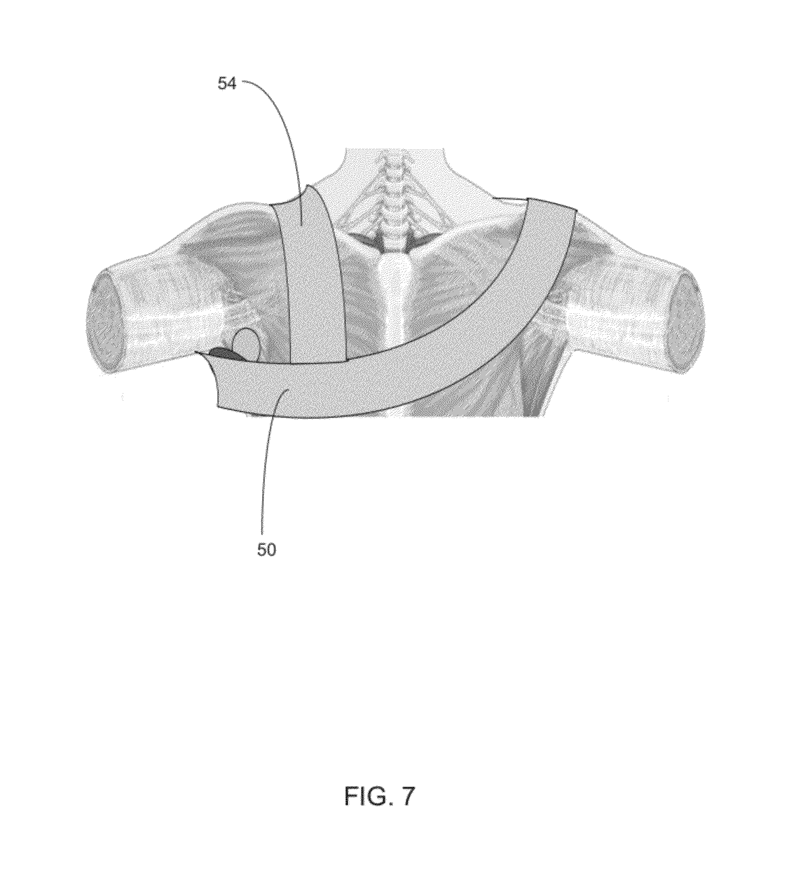

In another example, as shown in FIG. 7 the retention strap may be arranged to wrap around both the top of the opposing shoulder 50 and around the top of the ipsilateral (same side) shoulder 54, either in a two-strap or single strap construction. Similarly as above, the strap pattern may be stabilized, for example with the use of bridging hook-and-loop strips.

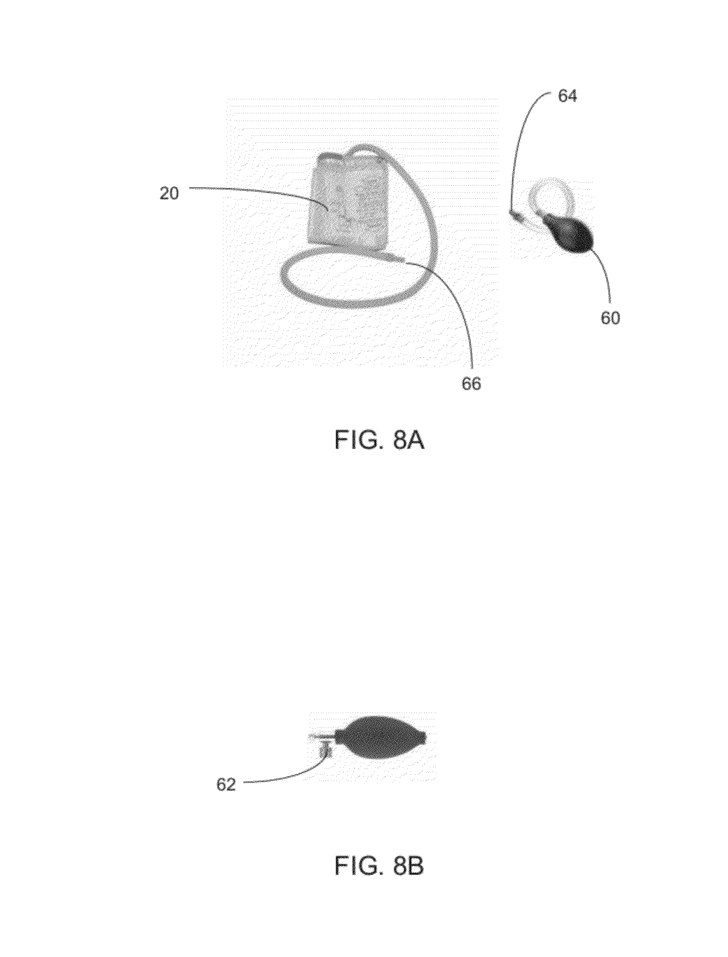

As shown in FIG. 8A, a bladder insufflator 60 may be permanently attached to, or detachable from the inflatable bladder 20, having a releasable one-way or check valve, or an adjustable air-release valve 62 (shown in FIG. 8B), as in a blood pressure cuff arrangement. The insufflator 60 may also be detachable from the bladder via an insufflator connector 64 and a bladder connector 66, as shown in FIG. 8A.

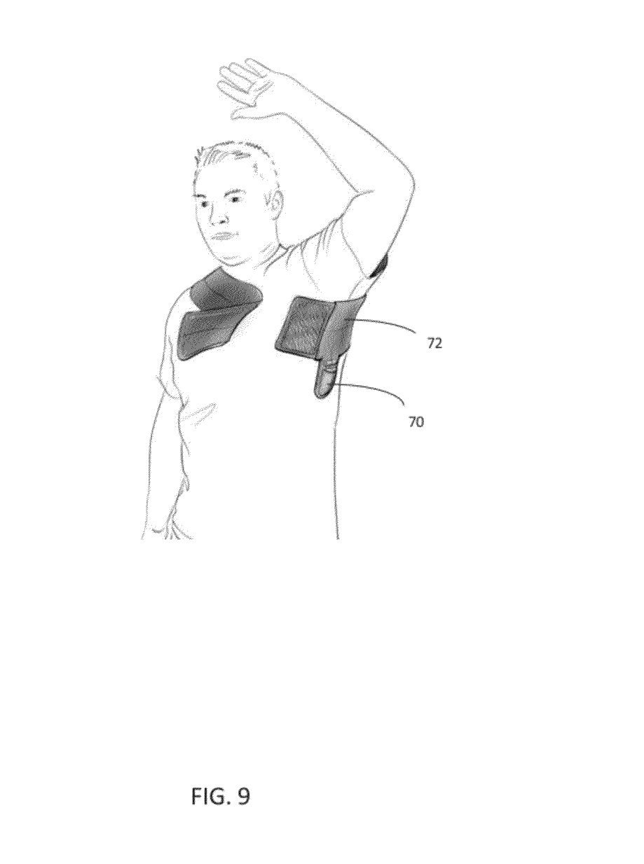

In another embodiment, the inflatable bladder, insufflator and strap may be incorporated into a single unit, as shown in FIG. 9. In this example, the insufflator 70 is formed as a component of the bladder structure 72, and has a relatively flat surface facing the torso.

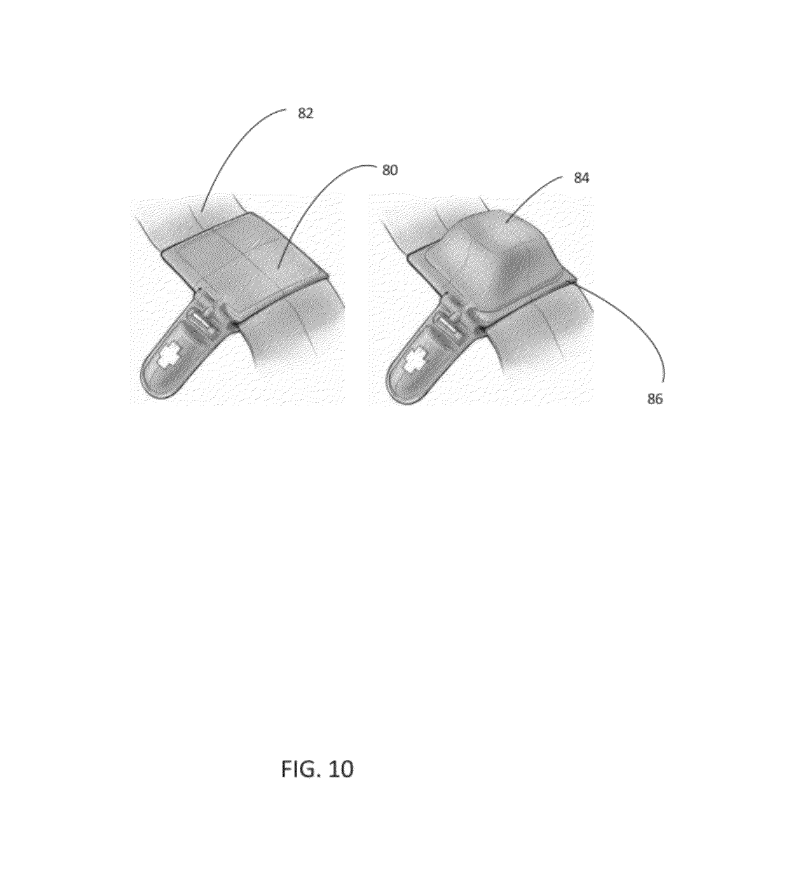

The bladder and its containment pouch may be constructed to have a generally square pyramidal or trapezoidal prism shape when inflated, as shown in FIG. 10. In other embodiments, the inflated shape of the bladder may take on a frustum (e.g., conical or pyramidal), in which the apex or tip is truncated by a planar or convex surface capable of applying relatively uniform pressure to the skin of the apex of the axilla. In deflated form, the bladder assembly 80 takes on a relatively flat, square or rectangular shape to conform with the attached strap 82. In this case, the inflated form factor 84 of the bladder may be shaped so as to be sufficient by itself to apply the appropriate distribution of force on the skin overlying the target artery without the need for a separate compressing mass. For placement in the axilla to occlude the axillary artery, for example, the apparatus may be used with or without a separate adjacently placed compressing mass.

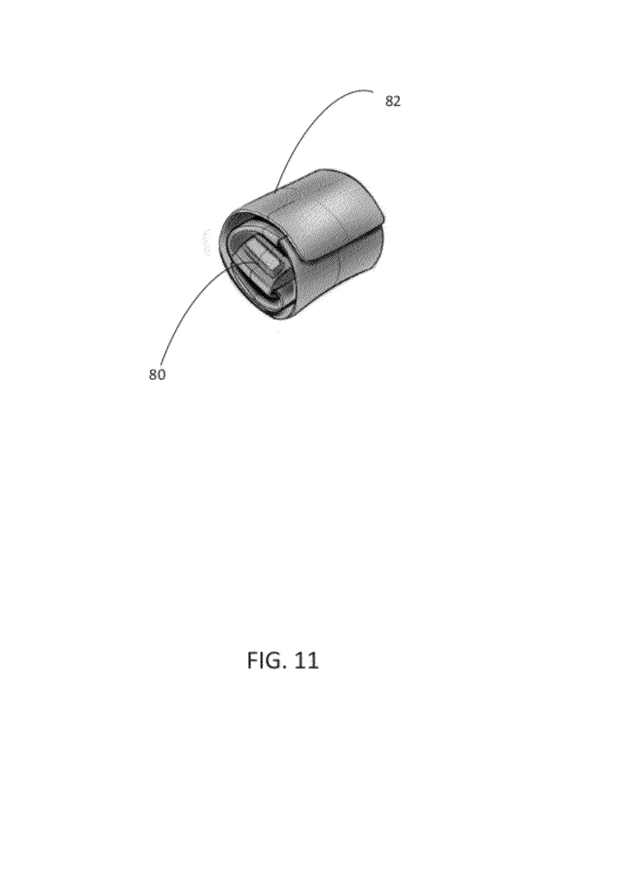

A platform or base plate 86 supporting the bladder assembly 80 may be constructed from semi-rigid or elastomeric material. In one aspect, it is rigid enough not to be deflected substantially by the adjacent inflating bladder, but it is flexible enough to bend upon arm movement when mounted on the torso. Some flexibility of the base plate may help to keep the bladder and/or occluder assembly from shifting positions during movement of the patient or the patient's arm. An advantage of this arrangement is that the strap 82 and bladder 80 assembly may be folded or rolled into a compact and easily stowable and transportable unit for use in field conditions, as shown in FIG. 11.

The inflation pressure of the bladder should be sufficient to compress the target artery, yet not so great as to excessively compress the surrounding tissues (such as the nerves of the brachial plexus, for example). This may require constant monitoring by an individual. Preferably, the inflation pressure in the bladder should be increased as soon as there is an indication of unwanted blood flow in the extremity under treatment. A peripheral pulse (e.g., of the brachial, radial or ulnar artery) may be manually monitored periodically. Alternatively, an oximetry sensor, may be placed on the extremity (e.g., on a digit) and monitored continuously by an electronic pulse oximeter.

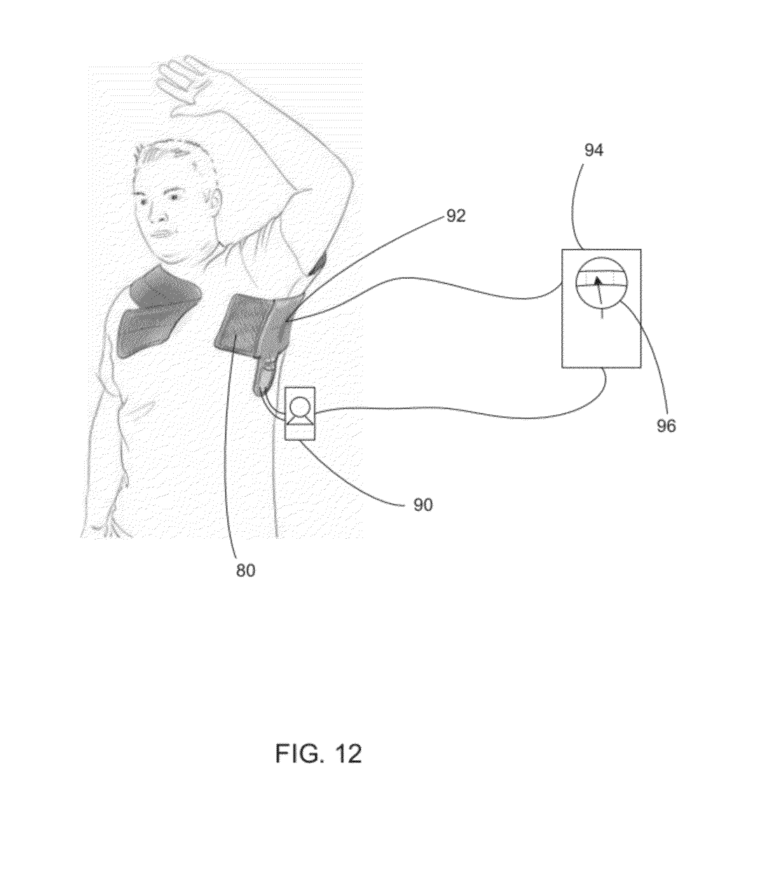

As shown schematically in FIG. 12, in an embodiment, the appropriate inflation pressure may be maintained automatically. This may include the use of a portable pump 90, a pressure sensor 92 either within the bladder 80 or externally adjacent to the bladder, and an electronic controller 94 to receive pressure readings from the sensor 92 (via wired or wireless transmission). The controller 94 may in turn control the operation of the pump 90 based on the pressure detected by the sensor 92. The inflation pressure range can be set by an operator through a user interface 96 on the controller, and the range adjusted to a patient's individual circumstances. The controller 94 may then be programmed to maintain the monitored pressure within the pre-set range by regulating (via wired or wireless transmission) the activity of the pump 90. An electromechanical pressure release valve may also be installed in the insufflation line or separately attached to the bladder, through which the controller may keep the bladder pressure from exceeding the pre-set range. Alternatively, a pulse sensor to detect the person's brachial, radial or ulnar pulse may be connected to the controller, which may regulate the amount of inflation of the bladder based on the strength, presence or absence of the pulse. In another embodiment, the controller may be programmed to receive a signal from a puke oximeter placed on a suitable location on the person's extremity, may regulate the amount of inflation of the bladder based on the strength of the oximetry signal.

1. An apparatus for applying pressure to an individual's axilla comprising:
An inflatable bladder mounted on flexible plate;
A flexible strap configured to support the plate in a position to allow an expandable surface of the inflatable bladder opposite the plate to face the axilla; wherein
The flexible strap is of a length sufficient to wrap around the individual's torso or over one of the individual's shoulders.

2. The apparatus of claim 1, further comprising a compressing mass adjacent the expandable surface of the inflatable bladder, wherein the compressing mass is of a size and shape configured to apply direct pressure to skin at an apex of the individual's axilla.

3. The apparatus of claim 1, wherein the expandable surface of the inflatable bladder is configured to have a size and shape upon inflation to apply direct pressure to skin at an apex of the individual's axilla.

4. The apparatus of claim 3, wherein the inflated shape of the expandable surface of the inflatable bladder is frusto-conical or frusto-pyramidal.

5. The apparatus of claim 2, wherein the compressing mass comprises a flexible component configured to conform to the shape of an apex of the individual's axilla.

6. The apparatus of claim 1, wherein the flexible strap comprises hook-and-loop material to allow an end of the flexible strap to be secured to one or more surfaces of the flexible strap.

7. A method of applying a compressive force to the axilla of an individual using a compressive device comprising an inflatable bladder mounted to a flexible base attached to a flexible strap, the method comprising:
facing an expandable surface of the bladder toward the individual's axilla;
wrapping the flexible strap around the individual's torso or over the individual's shoulder; and
inflating the inflatable bladder.

8. The method of claim 7, wherein the compressive device further comprises a compressing mass adjacent the expandable surface of the inflatable bladder, and wherein the method comprises facing the compressing mass toward the individual's axilla.

9. The method of claim 7, wherein the flexible strap comprises hook-and-loop material, and wherein the method comprises securing an end of the flexible strap to a portion of the hook-and-loop material after wrapping the flexible strap.

10. The method of claim 7, wherein the flexible strap comprises a buckle, and wherein the method comprises securing an end of the flexible strap by threading it through the buckle after wrapping the flexible strap.

11. The method of claim 7, wherein the method comprises inflating the inflatable bladder until blood flow through the axillary artery in the axilla is interrupted.

12. The method of claim 11, wherein interruption of blood flow in the axillary artery is detected by monitoring the brachial pulse, radial pulse, ulnar pulse or by pulse oximetry of a portion of the individual's upper extremity.

13. A system for reducing blood flow in a person's axilla by the application of pressure to skin and soft tissue in the axilla, comprising:
an inflatable bladder mounted on a flexible plate, an expandable portion of the bladder configured to expand toward an apex of the axilla;
a flexible strap to which the plate is attached, the strap being of a length sufficient to wrap around the person's torso or over the person's shoulder;
a releasable valve connected to the bladder, configured to connect to a manual or electromechanical pump for inflating the bladder; wherein
the inflatable bladder is configured to expand toward the apex of the axilla.

14. The system of claim 13, further comprising a compressing mass adjacent the expandable portion of the bladder, wherein the compressing mass is configured to generally conform to the shape of the apex of the axilla upon inflation of the bladder.

15. The system of claim 13, wherein the expandable surface of the inflatable bladder is configured to have a size and shape upon inflation to apply direct pressure to skin at an apex of the individual's axilla.

16. The system of claim 15, wherein the inflated shape of the expandable surface of the inflatable bladder is frusto-conical or frusto-pyramidal.

17. The system of claim 13, wherein the amount of expansion of the bladder is adjustable by operation of the releasable valve to release incremental amounts of air from the bladder.

18. The system of claim 13, wherein operation of the electromechanical pump is regulated to limit the inflation of the bladder to a pre-determined amount above the amount of inflation required to interrupt blood flow in an axillary artery within the axilla.

19. The system of claim 18, wherein the interruption of blood flow is detected by monitoring a brachial, radial or ulnar pulse, or by monitoring pulse oximetry in a portion of the person's upper extremity.

20. The system of claim 19, further comprising a controller configured to receive a signal from a pulse sensor or oximetry sensor, and configured to control the electromechanical pump and releasable valve to regulate the amount of inflation of the bladder.
